(19) United States
(12) Patent Application Publication
(10) Pub. No.: US 2022/0257571 A1
(43) Pub. Date: Aug. 18, 2022

(54) INHIBITOR OF MAP KINASE INTERACTING SERINE/THREONINE KINASE 1 (MNK1) AND MAP KINASE INTERACTING SERINE/THREONINE KINASE 2 (MNK2), CANCER THERAPY AND THERAPEUTIC COMBINATIONS

(71) Applicants: FUNPACIÓ HOSPITAL UNIVERSITARI VALL D'HEBRON - INSTITUT DE RECERCA, BARCELONA (ES); INSTITUT QUÍMIC DE SARRIÀ CETS FUNDACIÓ PRIVADA; CONSORCIO CENTRO DE INVESTIGACIÓN BIOMÉDICA EN RED, M.P., MADRID (ES)

(72) Inventors: Santiago RAMON Y CAJAL AGÜERAS, BARCELONA (ES); Stefan HÜMMER, BARCELONA (ES); Josep CASTELLVI VIVES, BARCELONA (ES); Elena MARTINEZ SÁEZ, BARCELONA (ES); José Ignacio BORRELL BILBAO, BARCELONA (ES); Jordi TEIXIDÓ CLOSA, BARCELONA (ES); Roger ESTRADA TEJEDOR, BARCELONA (ES); Elisabeth BOU PETIT, BARCELONA (ES); Vicente PEG CAMARA, BARCELONA (ES); Pedro Jesus GUIJARRO CARRILLO, BARCELONA (ES); Anna SANTAMARIA MARGALEF, BARCELONA (ES); Joan MOROTE ROBLES, SANT CUGAT DEL VALLÉS (ES); Leticia SUAREZ CABRERA, BARCELONA (ES)

(21) Appl. No.: 17/625,075

(22) PCT Filed: Jul. 9, 2020
(86) PCT No.: PCT/EP2020/069459

§ 371 Date: Jan. 5, 2022

(30) Foreign Application Priority Data
Jul. 10, 2019 (ES) P201930643

(51) Int. Cl. A61K 31/437; A61P 35/00; A61K 45/06

(52) U.S. Cl. CPC A61K 31/437; A61K 45/06; A61P 35/00

(57) ABSTRACT

The present invention describes 4,6-diphenyl-1H-pyrazolo[3,4-b]pyridin-3-amine (EB1), or a pharmaceutically acceptable salt thereof, as a selective MNK inhibitor for use in the treatment of cancers of an hormone-dependent organs, including triple-negative breast cancer, prostate cancer and other referable cancers with p-eIF4E overexpression due to increased MNK activity. The invention also includes particular therapeutic combinations including the 4,6-diphenyl-1H-pyrazolo[3,4-b]pyridin-3-amine.

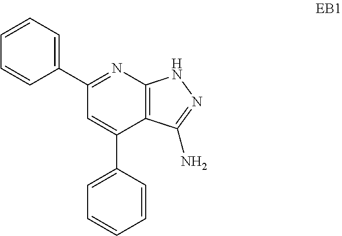 [SMILES: Nc1n[nH]c2nc(-c3ccccc3)cc(-c3ccccc3)c12]

This application claims the benefit of Spanish Patent Application P201930643 filed on Jul. 10, 2019.

### TECHNICAL FIELD

The present invention relates to the use of 4,6-diphenyl-1H-pyrazolo[3,4-b]pyridin-3-amine, or of a pharmaceutically acceptable salt thereof, as a selective MNK inhibitor with potential use in the treatment of cancers of hormone-dependent organs, such as breast cancer, including triple-negative breast cancer, and prostate cancer, and other referable cancers with p-eIF4E overexpression due to increased MNK activity.

### BACKGROUND ART

Breast cancer is the most common cancer in women worldwide and the second leading cause of cancer-related death. Despite global advances in cancer treatment, the fatality in some cancer subtypes with a poor prognosis has not improved substantially in recent years. Among the different breast cancer subtypes with a poor prognosis is triple-negative breast cancer (TNBC), characterised by the absence of estrogen receptor a (ER), progesterone receptor (PR) and human epidermal growth factor receptor 2 (HER-2) [Clin Cancer Res. 2007; 13:4429-4434]. This cancer subtype commonly develops with a relatively aggressive histopathological phenotype and tends to be diagnosed at younger ages than in ER-positive cases [Nat Rev Clin Oncol. 2016; 13:674-690; Oncologist. 2011; 16 Suppl 1:1-11; Breast Cancer Res. 2010; 12:212]. Since triple-negative tumours lack the primary targets of approved therapies, the cure thereof is currently limited to the application of surgery, radiotherapy and/or systemic chemotherapy. Therefore, despite advances in understanding the underlying biology of specific cancer subtypes, it is especially important to identify new therapeutic strategies for the treatment of these diseases.

Many studies have shown a significant clinical benefit of chemotherapy on TNBC in neoadjuvant, adjuvant and metastatic settings. Paradoxically, TNBC cases generally show better initial responses to chemotherapy than patients with other breast cancer subtypes [J. Am. Soc. Clin. Oncol. 2008; 26:1275-1281]. However, complete pathological responses are only achieved in 30-40% of cases at an early stage and patients who do not show this response level have a risk of death that is 12 times higher [Lancet Lond. Engl. 2014; 384:164-172; J. Am. Soc. Clin. Oncol. 2012; 30:1796-1804]. To improve the curing of cancer in this context, new strategies are being tested, such as immunotherapy, but its success to date is limited [Med. Oncol. Northwood Lond. Engl. 2017; 35:13]. Other subtypes which are estrogen positive have a better prognosis but a high risk of late recurrences. In fact, patients are treated with antiestrogens for many years. (J Clin Oncol. 2015 Mar. 10; 33(8):916-22). In this sense, various combinatorial approaches are being tried to optimize, including the combination with mnk inhibitors (Genes Dev. 2017 Nov. 15; 31(22):2235-2249)

Another type of cancer that develops desentizitation to approved treatments over time is prostate cancer. Prostate cancer is the most common malignancy and the second cause of cancer-related deaths among men in the western countries [CA Cancer J Clin. 2020 January; 70(1):7-30]. Most deaths occur after androgen-deprivation therapy failure, when the tumor evolves to castration resistant prostate cancer (CRPC). Despite initial response to second line anti-hormonal therapies (suchs as abiraterone and enzalutamide) patients usually develop resistance to these agents [BJU Int. 2016 February; 117(2):215-25]. Intratumoral androgen production, amplification, mutation or expression of consitutively active AR splice variantes lacking the C-terminal ligand-binding domain (being AR-V7 one of the most commonly found in CRPC patients) are some of the most important clinical mechanisms of resistance to anti-hormonal therapies [Cancer Treat Rev. 2017 June; 57:16-27]. As One of the main obstacles to the treatment of advanced prostate cancer is drug resistance, therapeutic combinatory strategies are desired.

Deregulation of protein synthesis is a common event in human cancer and, in particular, in resistance to chemotherapeutic agents. A key factor in translational control is the translation initiation factor 4E (eIF4E), the function of which is modulated by the MAP kinase-interacting serine/threonine-protein kinase 1, MNK1, and the MAP kinase-interacting serine/threonine-protein kinase 2, MNK2, through phosphorylation of a conserved serine (Ser209).

In recent years, eIF4E has been described as an independent prognostic factor associated with malignant progression and adverse prognosis in various tumours, including breast cancer, lung cancer, ovarian cancer, endometrial cancer, glioma and prostate cancer [Clin Transl Oncol. 2014; 16:937-1113]. Other groups have confirmed the prognostic importance of these factors in additional types of tumours, like colon cancer [Oncotarget. 2015; 6:24092-24104], nasopharyngeal carcinoma [PLoS ONE. 2014; 9:e89220], hepatocellular carcinoma [J Cancer Res Clin Oncol. 2016; 142:2309-2317], astrocytomas [J Neurooncol. 2016; 131:485-493], lung cancer [Virchows Arch. 2015; 467:667-673; Int J Clin Exp Pathol. 2015; 8:3955-3962] and melanoma [J Invest Dermatol. 2015; 135:1358-1367]. The worst prognosis has been associated with increased epithelial-mesenchymal transition in cells with p-eIF4E overexpression [Oncogene. 2015; 34(16):2032-2042] and a greater resistance to cellular oxidative stress, lack of nutrients and anti-tumour agents [PLoS ONE. 2015; 10(4):e0123352].

Studies have provided proof of concept that the deregulation of eIF4E-mediated translation initiation is an important step in oncogenic transformation and may contribute to tumour maintenance. Although phosphorylation is necessary for oncogenic transformation, it appears to be dispensable for normal development. Therefore, pharmacological MNK inhibitors may present an effective, non-toxic anti-cancer strategy, especially in combination with approved treatments. In summary, MNK1/2 have emerged as targets of interest for drug discovery in oncology, based on the anti-tumour effects observed in the experiments using RNA interference and the absence of adverse effects in double-knockout animals [Mol Cell Biol. 2004; 24(15):6539-6549]. However, the lack of selective MNK inhibitors has hampered the validation of drug targets and clinical development.

While the roles of MNKs in tumour development and progression have been well established, specific mono or dual MNK inhibitors with satisfactory levels of selectivity are still under development. Inhibitors like Cercosporamide [Cancer Biol Ther. 2015; 16(5):648-656] or CGP57380 [Cancer Res. 2011; 71:1849-1857], used for many years for the study of these kinases, are noted for their low selectivity [Biochem. J. 2007; 408(3):297-315; Curr Med Chem. 2017; 24(28):3025-3053].

MNKs belong to the serine/threonine-protein kinase family and are classified as members of the Ca²⁺/calmodulin-dependent kinases.

MNK kinases are present in two isoforms: MNK1 and MNK2. MNK1 is an inducible isoform, easily phosphorylated by ERK and p-38, whereas MNK2 has high basal activity.

While the overall structural architecture thereof resembles other protein kinases, MNKs contain several unusual elements [Oncotarget. 2012; 3:118-131; Chem Biol. 2014; 4:441-452]. These differences open up the possibility of developing highly selective inhibitors. Structural studies carried out in recent years have proposed different types of interactions between the ligands and active kinase sites: Type I (active kinase conformation), Type II (inactive kinase conformation), Type III (allosteric inhibitors) and Type VI (irreversible inhibitors) [Curr Med Chem. 2017; 24(28):3025-3053].

Recently, the first two MNK inhibitors developed by Effector Therapeutics and Bayer AG (eFT508 and BAY 1143269, respectively) have entered clinical trials in oncology [J Med Chem. 2018; 61(8):3516-3540; Cancer Lett. 2017; 390:21-29]. According to the classification of MNK inhibitors, both are Type I inhibitors and act competitively for ATP. eFT508 has been successfully applied in the treatment combined with an mTOR inhibitor (everolimus) in large B-cell lymphomas, and BAY1143269 has been combined with taxanes in the treatment of non-small cell lung cancer. While these results confirm that MNK1/2 are a valuable target for preventing adaptation to approved treatments, the ATP-competitive mode of action may hinder wider use of these inhibitors in other types of cancers. In this regard, no results regarding the treatment of TNBC with both Type I inhibitors have been published.

In recent years, various research groups have been involved in the development of MNK1/2 inhibitors using various heterocyclic systems as a base structure. In particular, relevant to this patent application are those works that have used 1H-pyrazolo[3,4-b]pyridin-3-amine heterocyclic systems as the base structure for developing kinase inhibitors. Thus, US 2010/0113415 describes the use of said heterocycles as EphA4 receptor tyrosine kinase inhibitors which are useful in the treatment of neurological and neurodegenerative disorders, cancer and other conditions regulated by EphA4 receptor tyrosine kinase signalling. The two tested compounds in US 2010/0113415, labelled as Examples 63 and 67 provided a demosntrated effect as EphA4 receptor tyrosine kinase inhibitors. These assays do not illustrate or confirm, however, that they are useful in the treatment of cancers of hormone-dependent organs, such as breast cancer (including TNBC), ovarian cancer, endometrial cancer and prostate cancer, much less than all other listed compounds imply the said inhibitory effect of EphA4 receptor tyrosine kinase.

Furthermore, WO 2011/019780 claims the use of these heterocyclic systems as inhibitors of protein kinase enzymes for the treatment of allergic disorders, autoimmune and/or inflammatory diseases, and cancers. None of these patents, however, mention MNK1/2 or eIF4E phosphorylation.

Lastly, WO 2015/004024 describes 1H-pyrazolo[3,4-b]pyridin-3-amine systems with a very bulky heterocyclic substituent at position C5 of the pyrazolopyridine system. In this case, the resulting systems are indeed claimed as inhibitors of MNK1 kinase and/or MNK2 kinase, mentioning their connection with eIF4E and their involvement in the treatment of breast cancer.

Thus, although many efforts have been done for providing effective cancer therapies, there is still a need of alternative ones and particularly for those cancer types that, in some way, become resistant or refractive to common therapies.

### SUMMARY OF INVENTION

Present invention results from the surprising MNK1/2 inhibitory effect of one of the simplest 1H-pyrazolo[3,4-b]pyridin-3-amine derivatives: compound 4,6-diphenyl-1H-pyrazolo[3,4-b]pyridin-3-amine, (named in this description as EB1).

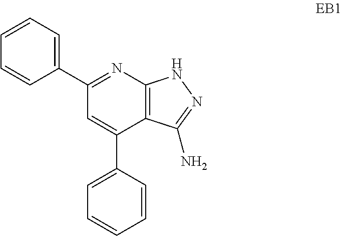 [SMILES: Nc1n[nH]c2nc(-c3ccccc3)cc(-c3ccccc3)c12]

Said compound was first described in DE 2160780 (1973) as an intermediate for the synthesis of dyes and in other articles and patents as a synthesis intermediate (such as for example in US 2010/0113415). However, the possible biological activity thereof in connection with MNK1/2 has apparently not been described yet.

Inventors surprisingly found that EB1 could selectivily inhibit MNK1/2 (enzymatic ICso of 0.7 μM and an in vitro EC₅₀ of approximately 1.5 μM in triple-negative breast cancer cells (MDA-MB-231)). This inhibition was, in addition, a non-ATP competitive MNK1/2 inhibition, a genuine feature in relation with the other MNK1/2 inhibitors known and commercially available nowadays. Moreover, as MNK1/2 inhibitor it was no toxic when tested on normal cells.

Selectivity of EB1 was tested in a panel of 320 kinases which included EphA4 receptor tyrosine kinase. Although EB1 is mentioned as intermediate for the synthesis of other active compounds with inhibitory effect of this receptor in US 2010/0113415, no activity for EB1 could be inferred from the assays disclosed in this prior art document. Indeed, in an inhibitory test of EphA4 carried out with EB1 in this kinase panel using the same protocol, only an inhibition of 10% was obtained at 1 uM while the inhibition of MNK1 was 48%, so it is clear the virtual lack of inhibitory activity of EB1 against EphA4.

Thus, in a first aspect, the invention relates to 4,6-diphenyl-1H-pyrazolo[3,4-b]pyridin-3-amine or a pharmaceutically or veterinary acceptable salt thereof, for use in the treatment of a cancer of an hormone-dependent organ selected from breast cancer, prostate cancer, ovarian cancer and endometrial cancer.

This aspect may also be formulated as a method of treatment and/or prevention of a cancer of an hormone-dependent organ selected from breast cancer, prostate cancer, ovarian cancer and endometrial cancer, which comprises administering to a mammal subject in need thereof, a therapeutically effective amount of EB1, or a pharmaceutically or veterinary acceptable salt thereof, in a subject in need thereof including a human subject.

It also forms part of the invention the use of a therapeutically effective amount of EB1, or a pharmaceutically or veterinary acceptable salt thereof, for the preparation of a medicament for the treatment and/or prevention of a cancer of an hormone-dependent organ selected from breast cancer, prostate cancer, ovarian cancer and endometrial cancer.

Results from other derivatives studied with various substituents included in the phenyl rings at positions C4 and C6 of the 1H-pyrazolo[3,4-b]pyridin-3-amine system have presented activities lower than those of EB1. As will be illustrated in more detail in the description below, EB1 inhibits eIF4E phosphorylation with in vitro EC₅₀˜1.5 μM with almost complete inhibition as of 2.5 μM and the effect is fast and lasts for at least 72 h; the inhibitory effect seems to be due to the direct inhibition of MNKs; and noteworthy, EB1 shows excellent selectivity for MNK1/2 in relation with other kinases.

Inventors have also surprisingly found that when EB1 is used in combination with certain chemotherapeutic and/or immunotherapeutic compounds or agents of those commonly used in the treatment of these cancers of hormone-dependent organs, the observed therapeutic effect is synergistically increased in terms of one or more of reduced cell growth and induction of apoptosis.

Thus, a second aspect of the invention relates to combinations comprising:

a) a therapeutically effective amount of EB1 or a pharmaceutically or veterinary acceptable salt thereof; and
b) a therapeutically effective amount of one or more chemotherapeutic or immunotherapeutic compounds selected from the group consisting of doxorrubicin, tamoxifen, enzalutamide, docetaxel, cisplatin, lapatinib, inhibitors of the programmed cell-death protein 1 and its ligand (PD-1/PD-L1), and combinations thereof.

These combinations including EB1 are proposed, without being bound to theory, because EB1 as MNK inhibitor makes possible to sensitise tumour cells to common therapies, to which common therapies certain subjects or population have developed resistance. Resistance to a cancer therapy is in part developed, as indicated above, due to the deregulation of protein synthesis in response to cell stress caused by toxic agents, such as a compound for treating cancer as a cell protection mechanism. The MNKs are required under cellular stress (activated by the stress pathways), which often prevents classical therapies to be effective. Targeting MNKs with EB1 or a salt thereof sensitizes the cells to standard of care treatments that fail due to the activation of stress pathways.

The compounds in combinations of the invention may be formulated in different types of compositions/kits of parts. Thus, a third aspect of the invention relates to a single pharmaceutical or veterinary composition which comprises:

a) a therapeutically effective amount of EB1 or a pharmaceutically acceptable salt thereof; and
b) a therapeutically effective amount of one or more chemotherapeutic or immunotherapeutic compounds selected from the group consisting of doxorrubicin, tamoxifen, enzalutamide, docetaxel, cisplatin, lapatinib, inhibitors of the programmed cell-death protein 1 and its ligand (PD-1/PD-L1), and combinations thereof, together with one or more pharmaceutically or veterinary acceptable excipients or carriers.

A fourth aspect of the invention relates to a package or kit of parts comprising:

i) a first pharmaceutical or veterinary composition which comprises a therapeutically effective amount of EB1, or a pharmaceutically or veterinary acceptable salt thereof, together with one or more pharmaceutically or veterinary acceptable excipients or carriers;
ii) a second pharmaceutical or veterinary composition which comprises a therapeutically effective amount of one or more chemotherapeutic or immunotherapeutic compounds selected from the group consisting of doxorrubicin, tamoxifen, enzalutamide, docetaxel, cisplatin, lapatinib, inhibitors of the programmed cell-death protein 1 and its ligand (PD-1/PD-L1), and combinations thereof, together with one or more pharmaceutically or veterinary acceptable excipients or carriers;
iii) instructions for the use in combination of i) and ii);
wherein the first and second compositions are separate compositions.

Finally, precisely due to the selectivity for MNK1/2, it is also herewith disclosed the non-therapeutically use of 4,6-diphenyl-1H-pyrazolo[3,4-b]pyridin-3-amine, or of a pharmaceutically or veterinary acceptable salt thereof, as a selective, non-cytotoxic inhibitor of MAP kinase-interacting serine/threonine-protein kinase 1 (MNK1) and MAP kinase-interacting serine/threonine-protein kinase 2 (MNK2). This use is of interest as inhibitor of MNK1/2 in an isolated sample comprising mammal cells (i.e. isolated biopsies of tumour tissue, stablished cell lines, etc.) with the aim testing therapeutic combinations, or as reagent in biochemistry assays in which isolated mammal cells are used to study mechanistic cell processes, metabolic pathways or to test substances in screening methods. The inhibition of MNK1 and MNK2 by means of EB1 causes the inhibition of eIF4E phosphorylation.

### BRIEF DESCRIPTION OF DRAWINGS

To complement the description that is being made and for the purpose of helping to better understand the features of the invention, a set of drawings is attached as an integral part of said description, wherein the following has been depicted, with an illustrative and non-limiting character:

FIG. 1 shows an example of the titration curves of EB1 in MDA-MB-231 cells which show no effect on upstream proteins and which demonstrate selective MNK inhibition. The results were equivalent at 24 h and 48 h. Final concentration of dimethyl sulfoxide (DMSO) 0.5%. CGP57380 (CGP) is used as a positive control and DMSO as a negative control.

FIG. 2 shows titration curve EB1 in MCF10 cells. Final concentration of DMSO 0.5%. CGP57380 (CGP) is used as a positive control and DMSO as a negative control.

FIG. 3 shows proliferation curves for EB1 in MDA-MB-231 and MCF10 cells. CGP57380 (CGP) and EFT508 are used as positive controls and DMSO as a negative control. Norm. Abs. is the normalized absorbance and T(h) is the time in hours.

FIG. 4 shows the MDA-MB-231 cell cycle treated with EB1. 100 nM doxorubicin is used as a positive control and DMSO as a negative control.

FIG. 5 shows a kinome for EB1.

FIG. 6 shows a titration curve of EB1 in A375M, MV4-11 and MDA-MB-468 cells. Final concentration of DMSO 0.5%. CGP57380 (CGP) is used as a positive control and DMSO as a negative control.

FIG. 7 shows that the combination of doxorubicin (DOXO) with EB1 increases the efficacy of the chemotherapeutic drug (B) and reverses the increase in p-eIF4E caused by cellular stress related to treatment with doxorubicin (A).

FIG. 8 shows that treatment of IMR90 cells, non-transformed fibroblasts, does not cause significant cell growth arrest (Figure A, where RCG is relative cell growth) and does not induce cell death (Figure B, % Apopototic cells). In contrast, in all tested breast cancer cell lines (MDA-MB231, MCF7 and MDA-MB468) cell growth is inhibited in a dose dependent manner. DMSO means dimethylsulfoxide; CDDP is cisplatin; RCG is relative cell growth

FIG. 9 shows tha EB1 treatment of MCF7 cells results in increased sensitivity to tamoxifen. CGP means CGP57380.

FIG. 10 shows thatEB1 treatment reduces levels of PD-L1 in the human breast cancer cell line MDA-MB231. At comparable inhibition rates of eIF4E phosphorylation, EB1 is equally potent or superior to the MNK inhibitor eFT508 and CGP57380.

FIG. 11 shows cell viability under combined inhibition of AR and eIF4E phosphorylation. (A) Dose dependent inhibition of eIF4E phosphorylation after 24 h EB1 treatment in 22Rv1. Synergistic effect of the EB1 and Enzalutamide with 22Rv1 (B-F) cell viability assays. (B) Number of viable cells after 72 h treatment with pairwise combinations reported as percentage relative to control. (E) Cell viability data presented as a grid displaying the percentage of affected cells by each pairwise combination of drug doses. (C) Drug combinations with the strongest inhibition in cell viability (bars; mean±SEM; n=3. *p<0.05, **p<0.01 two-tailed Student's test). (F) Combination index (CI) presented as a grid for each pairwise combination of drug doses calculated by the Chou-Talalay method at non-constant ratio. (D) Fraction affected vs. combination index (CI) plots.

FIG. 12 shows that dual inhibition of AR and eIF4E phosphorylation induces cell death via apoptosis in cell lines 22Rv1 (A) cells were treated either with vehicle (DMSO), enzalutamide, EB1 or a combination (Combo) of both with the indicated concentrations for 72 h. (B) Apoptosis assays of 22Rv1 cell line after treatment with the indicated concentrations of Enzalutamide, EB1 and the combination of both for 72 h, quantified by analysis of fragmented/condensed chromatin after Propidium Iodide staining. Data are mean of two technical replicates from one experiment ±SEM ***p<0.001 two-tailed Student's test

### DETAILED DESCRIPTION OF THE INVENTION

Unless otherwise stated, all terms as used in this application shall be understood in their ordinary meaning as known in the art. Other more specific definitions for certain terms as used in the present application are as set forth below and are intended to apply uniformly throughout the specification and claims unless an otherwise expressly set out definition provides a broader meaning.

As used herein, the indefinite articles “a” and “an” are synonymous with “at least one” or “one or more.” Unless indicated otherwise, definite articles used herein, such as “the,” also include the plural of the noun.

The term “cancer of hormone-dependent organs” includes cancers of organs that are dependent of hormones for their function, in particular of sex hormones (androgens, estrogens, progestogens) for their function or even that secrete such sex hormones, as reproductive and sexual organs are, including breast, ovarian, endometrium, prostate and testicles. Within these cancers of hormone-dependent organs there are included: (a) those hormone-dependent cancers, thus cancers that are dependent on a hormone for growth and/or survival, also called “hormone-sensitive”; (b) cancers that become independent of hormones during the disease progression; and (c) hormone-independent cancers but with origin in hormone-dependent organs, such as TNBC.

For “inhibitors of the programmed cell-death protein 1 and its ligand (PD-1/PD-L1)” is to be understood those compounds that. The section of examples includes assays to determine the inhibitory effect, and the skilled person in the art knows also how to test this inhibitory effect.

The expression “therapeutically effective” as used herein throughout this description, refers to the amount of a compound or combination of compounds that, when administered, is enough to prevent development of, or alleviate to some extent, one or more of the symptoms of the disease which is addressed. In this particular description, it is the amount of a compound, combination of compounds, or composition that produces a desired therapeutic effect in a subject, such as treating cancer, in particular cancer of hormone. dependent organs, including breast cancer and prostate cancer. The precise therapeutically effective amount is an amount of the composition that will yield the most effective results in terms of therapeutic efficacy in a given subject. The specific dose of the EB1 to obtain a therapeutic benefit may vary depending on the particular circumstances of the individual patient including, among others, the size, weight, age and sex of the patient, the nature and stage of the disease, the aggressiveness of the disease, and the route of administration. The specific dose of EB1 to obtain a therapeutic benefit when administered in the herewith disclosed combinations, compositions or kit of parts, may vary in relation with the specific dose of the compound used as single active agent.

There is no limitation on the type of salt of the compounds EB1 that can be used, provided that these are pharmaceutically or veterinary acceptable when they are used for the therapeutic purpose. The term “pharmaceutically or veterinary acceptable salts”, embraces salts commonly used to form alkali metal salts and to form addition salts of free acids or free bases. The preparation of pharmaceutically or veterinary acceptable salts of the compounds EB1 can be carried out by methods known in the art.

For instance, they can be prepared from the parent compound, by conventional chemical methods. Generally, such salts are, for example, prepared by reacting the parent compound EB1 with a stoichiometric amount of the appropriate pharmaceutically or veterinary acceptable base or acid in water or in an organic solvent or in a mixture of them. The compound EB1 and their respective salts may differ in some physical properties but they are equivalent for the purposes of the present invention.

The compound EB1 may be as an amorphous or crystalline solid form either as free solvation compound or as solvates (e.g. hydrates) and it is intended that all forms are within the scope of the present invention for the purposed therapeutic use and for the disclosed combinations. Methods of solvation are generally known within the art. In general, the solvated forms with pharmaceutically, or veterinary acceptable solvents such as water, ethanol and the like are equivalent to the unsolvated form for the purposes of the invention. In particular, the compound EB1 is used according to this description as an amorphous solid.

In all embodiments of the invention referring to the EB1, the pharmaceutically or veterinary acceptable salts thereof are always contemplated even if they are not specifically mentioned.

The expression “pharmaceutically or veterinary acceptable excipients or carriers” refers to pharmaceutically or veterinary acceptable materials, compositions or vehicles. Each component must be pharmaceutically or veterinary acceptable in the sense of being compatible with the other ingredients of the pharmaceutical or veterinary composition. It must also be suitable for use in contact with the tissue or organ of humans and animals without excessive toxicity, irritation, allergic response, immunogenicity or other problems or complications commensurate with a reasonable benefit/risk ratio.

The phrases “patient” and “subject” are used interchangeably herein, and they relate to any mammal, in particular they relate to human.

When in this description the expression “treatment of cancer” is used it relates to any of the treatment and/or the prevention of the indicated cancer, although not specifically stated.

As previously indicated, a first aspect of the invention relates to 4,6-diphenyl-1H-pyrazolo[3,4-b]pyridin-3-amine (EB1) or a pharmaceutically acceptable salt thereof, for use in the treatment of a cancer of an hormone-dependent organ selected from breast cancer, prostate cancer, ovarian cancer and endometrial cancer.

All these cancers in which 4,6-diphenyl-1H-pyrazolo[3,4-b]pyridin-3-amine or a pharmaceutically acceptable salt thereof is proposed, are cancers also referable by inhibition of MNK1 and MNK2, in which over-expression of p-eIF4E takes place. Indeed, other cancers in which such p-eIF4E overexpression takes place, and also disclosed herewith are selected from the group consisting of lung cancer, colon cancer, melanoma, sarcoma, leukaemia, lymphomas and brain (i.e. glioma) tumours. In all these cancers that can be treated with EB1 or a pharmaceutically or veterinary acceptable salt thereof, p-eIF4E overexpression is induced by treatments selected from the group consisting of chemotherapy, radiotherapy and immunotherapy.

Within the cancers of an hormone-dependent organs, the hormone-dependent cancers are commonly treated mainly by blocking interaction of the hormones with the corresponding cell receptors in tumour cells. This makes that stress pathway activates and leads cancer cells to evade the treatment (resistance to treatment), because said tumour cells become no sensitive. These resistant cancers are those that have become independent of hormones during the disease progression and their treatment is challenging. Finally, the hormone-independent cancers that origin in hormone-dependent organs, such as TNBC, are cancers that as such do not respond to hormone or antihormone therapy, because they lack hormone receptors or express receptors that lack the hormone or antihormone binding domain.

Thus, in a particular embodiment of the first aspect, EB1 or a pharmaceutically or veterinary salt thereof, is for use in the treatment of these cancers of hormone-dependent organs in a population of subjects that have developed resistance, or that are as such resistant, to targeted chemotherapy and/or resistant to immunotherapy commonly used for these cancers.

For “targeted chemotherapy” is to be understood that the cancer is treated using drugs to target specific genes and proteins that are involved in the growth and survival of cancer cells. Targeted therapy can affect the tissue environment that helps a cancer grow and survive or it can target cells related to cancer growth, like blood vessel cells.

This is noteworthy in the case of TNBC, as mentioned, characterised by the absence ER, PR and HER-2, and thus only cured by means of surgery, radiotherapy and/or systemic chemotherapy. Assurance that the systemic chemotherapy in TNBC is effective, or resistance is not developed, can be attained (see examples below) if EB1 or a pharmaceutically or veterinary salt thereof is used in this particular breast cancer.

Most breast carcinomas are estrogen positive an have better prognosis but high risk of late recurrences. In fact, patients are treated with antiestrogens for many years. And new pharmacological approaches are being studfied, including the combination with mnk inhibitors

Also noteworthy is for prostate cancer, specially in the context of castration resistance, where the tumor cells are no longer controlled by androgen suppresion but the AR remains playing a critical role, in many cases due to the acquisition of alterations such as amplifications, activating mutations on the ligand binding domain (LBD) and splice variants, particularly the consitutively active variants that lack the LBD (i.e. AR-V7). Such AR modifications allow its activation in the absence of hormones and the development of clinical resistance to potent AR-targeted therapies (i.e. enzalutamide, AR-inhibitor that binds to the AR-LBD).

Therefore, EB1 or its salts are proposed for use in the treatment of cancers of hormone-dependent organs in patients non-sensitive to common therapies and/or in patients that, after the treatment with the commonly used therapies, develop resistance due to a cell phenotype change (deregulation of protein synthesis in response to cell stress). Advantadgeously, this approach allows to re-sensitize those patients that have evolved to a non-respondent profile, and make the common targeted therapy effective again.

Thus, in another particular embodiment of the first aspect, EB1 is for use in the treatment of a cancer of an hormone-dependent organ selected from breast cancer and prostate cancer.

In another more particular embodiment, optionally in combination with any embodiment above or below, the breast cancer is the triple negative breast cancer (TNBC).

Also in another more particular embodiment, optionally incombination with any embodiment above or below, the cancer of an hormone-dependent organ is prostate cancer.

In yet another particular embodiment of the first aspect, the 4,6-diphenyl-1H-pyrazolo[3,4-b]pyridin-3-amine (EB1) or a pharmaceutically acceptable salt thereof for use as indicated above, is such that the treatment comprises administering 4,6-diphenyl-1H-pyrazolo[3,4-b]pyridin-3-amine (EB1) or a pharmaceutically or veterinary acceptable salt thereof, in combination with one or more compounds selected from compounds for chemotherapy and compounds for immunotherapy.

In a more particular embodiment when EB1 or a salt thereof is for use in combination with other compounds for chemotherapy or immunotherapy, and optionally in combination with one or more features of the various embodiments described above or below throughout all the description, the treatment comprises the simultaneous, concurrent, separate or sequential administration of: (a) EB1, or a pharmaceutically or veterinary acceptable salt thereof; and (b) compounds selected from compounds for chemotherapy and compounds for immunotherapy.

In another particular embodiment of the first aspect, the chemotherapeutic compounds are selected from doxorubicin, tamoxifen, enzalutamide, docetaxel, cisplatin, lapatinib and combinations thereof.

In yet another more particular embodiment EB1, or a pharmaceutically or veterinary acceptable salt thereof, is for use as disclosed in the first aspect in combination with doxorubicin.

In yet another more particular embodiment EB1, or a pharmaceutically or veterinary acceptable salt thereof, is for use as disclosed in the first aspect in combination with tamoxifen.

In yet another more particular embodiment EB1, or a pharmaceutically or veterinary acceptable salt thereof, is for use as disclosed in the first aspect in combination with enzalutamide.

In yet another more particular embodiment EB1, or a pharmaceutically or veterinary acceptable salt thereof, is for use as disclosed in the first aspect in combination with docetaxel.

Also in another particular embodiment of the first aspect, optionally in combination with any embodiment above or below, the compounds for immunotherapy comprise inhibitors of the programmed cell-death protein 1 and its ligand (PD-1/PD-L1).

Examples of PD1 inhibitors include the approved Pembrolizumab, Nivolumab, Cemiplimab. Other examples still in experimental phase are Partalizumab, Camrelizumab (SHR1210), Sintilimab (1B1308), Tislelizumab (BGB-A317), Toripalimab (JS 001), Dostarlimab (TSR-042, WBP-285), INCMGA00012 (MGA012), AMP-224 and AMP-514 (MED10680).

Examples of PD-L1 inhibitors include the approved Atezolizumab, Avelumab, Durvalumab. Other examples still in experimental phase are KN035, CK-301, AUNP12, CA-170, and BMS-986189.

Present invention relates in a second aspect to particular new combinations of chemotherapeutic or immunotherapeutic compounds with EB1, or a pharmaceutically or veterinary acceptable salt thereof, such as the combinations comprising:

a) a therapeutically effective amount of EB1 or a pharmaceutically or veterinary acceptable salt thereof; and
b) a therapeutically effective amount of one or more chemotherapeutic or immunotherapeutic compounds selected from the group consisting of doxorrubicin, tamoxifen, enzalutamide, docetaxel, cisplatin, lapatinib, inhibitors of the programmed cell-death protein 1 and its ligand (PD-1/PD-L1), and combinations thereof.

In a particular embodiment of this second aspect, the combination comprises a therapeutically effective amount of EB1, or a pharmaceutically or veterinary acceptable salt thereof, and a therapeutically effective amount of one or more of enzalutamide and docetaxel. These combinations are in particular for use in the treatment of prostate cancer.

In also another particular embodiment of the second aspect, the combination comprises a therapeutically effective amount of EB1, or a pharmaceutically or veterinary acceptable salt thereof, and a therapeutically effective amount of one or more of doxorrubicin and tamoxifen. These combinations are in particular for use in the treatment of breast cancer, and more in particular the triple negative breast cancer.

In also another particular embodiment of the second aspect, the combination comprises a therapeutically effective amount of EB1, or a pharmaceutically or veterinary acceptable salt thereof, and a therapeutically effective amount of one or more of an inhibitor of PD-1 or PD-L1.

Another aspect of the invention is a single pharmaceutical or veterinary composition which comprises:

a) a therapeutically effective amount of EB1, or a pharmaceutically or veterinary acceptable salt thereof; and
b) a therapeutically effective amount of one or more chemotherapeutic or immunotherapeutic compounds selected from the group consisting of doxorrubicin, tamoxifen, enzalutamide, docetaxel, cisplatin, lapatinib, inhibitors of the programmed cell-death protein 1 and its ligand (PD-1/PD-L1), and combinations thereof, together with one or more pharmaceutically or veterinary acceptable excipients or carriers.

In a particular embodiment of this single pharmaceutical or veterinary composition, it comprises a therapeutically effective amount of EB1, or a pharmaceutically or veterinary acceptable salt thereof, and a therapeutically effective amount of doxorrubicin.

In another particular embodiment of the single pharmaceutical or veterinary composition, it comprises a therapeutically effective amount of EB1, or a pharmaceutically or veterinary acceptable salt thereof, and a therapeutically effective amount of tamoxifen.

In another particular embodiment of the single pharmaceutical or veterinary composition, it comprises a therapeutically effective amount of EB1, or a pharmaceutically or veterinary acceptable salt thereof, and a therapeutically effective amount of enzalutamide.

In another particular embodiment of the single pharmaceutical or veterinary composition, it comprises a therapeutically effective amount of EB1, or a pharmaceutically or veterinary acceptable salt thereof, and a therapeutically effective amount of docetaxel.

In another particular embodiment of the single pharmaceutical or veterinary composition, it comprises a therapeutically effective amount of EB1, or a pharmaceutically or veterinary acceptable salt thereof, and a therapeutically effective amount of an inhibitor of the programmed cell-death protein 1

In another particular embodiment of the single pharmaceutical or veterinary composition, it comprises a therapeutically effective amount of EB1, or a pharmaceutically or veterinary acceptable salt thereof, and a therapeutically effective amount of an inhibitor of the ligand of the programmed cell-death protein 1.

In another particular embodiment, optionally in combination with one or more features of the various embodiments described above or below throughout all the description, the invention also relates to a package or kit of parts comprising:

i) a first pharmaceutical or veterinary composition which comprises a therapeutically effective amount of EB1, or a pharmaceutically or veterinary acceptable salt thereof, together with one or more pharmaceutically or veterinary acceptable excipients or carriers;
ii) a second pharmaceutical or veterinary composition which comprises a therapeutically effective amount of one or more chemotherapeutic or immunotherapeutic compounds selected from the group consisting of doxorubicin, tamoxifen, enzalutamide, docetaxel, cisplatin, lapatinib, inhibitors of the programmed cell-death protein 1 and its ligand (PD-1/PD-L1), and combinations thereof, together with one or more pharmaceutically or veterinary acceptable excipients or carriers;
iii) instructions for the use in combination of i) and ii);
wherein the first and second compositions are separate compositions.

The election of the pharmaceutical or veterinary formulation will depend upon the nature of the active compound and its route of administration. Any route of administration may be used, for example oral, parenteral and topical administration.

For example, the pharmaceutical or veterinary composition may be formulated for oral administration and may contain one or more physiologically compatible carriers or excipients, in solid or liquid form. These preparations may contain conventional ingredients such as binding agents, fillers, lubricants, and acceptable wetting agents.

The pharmaceutical or veterinary composition may be formulated for parenteral administration in combination with conventional injectable liquid carriers, such as water or suitable alcohols. Conventional pharmaceutical or veterinary excipients for injection, such as stabilizing agents, solubilizing agents, and buffers, may be included in such compositions. These pharmaceutical or veterinary compositions may be injected intramuscularly, intraperitoneally, or intravenously.

The pharmaceutical composition may be formulated for topical administration. Formulations include creams, lotions, gels, powders, solutions and patches wherein the compound is dispersed or dissolved in suitable excipients.

The pharmaceutical compositions may be in any form, including, among others, tablets, pellets, capsules, aqueous or oily solutions, suspensions, emulsions, or dry powdered forms suitable for reconstitution with water or other suitable liquid medium before use, for immediate or retarded release.

The appropriate excipients and/or carriers, and their amounts, can readily be determined by those skilled in the art according to the type of formulation being prepared.

It forms part of the invention the combination, single pharmaceutical or veterinary composition, package or kit of parts comprising a therapeutically effective amount of EB1, or a pharmaceutically or veterinary salt thereof; and one or more of the chemotherapeutic or immunotherapeutic compounds as listed above, for use in the treatment and/or prevention of cancer in a subject in need thereof. In particular, they are for use in cancers selected from the group consisting of breast cancer, lung cancer, ovarian cancer, endometrial cancer, colon cancer, prostate cancer, melanoma, sarcoma, leukaemia, lymphomas and brain tumours, including glioma. More in particular, they are for use in the treatment of cancers of hormone-dependent organs in a subject in need thereof. More in particular, for use in breast and prostate cancer in a subject in need thereof.

Thus, any of the combinations selected from the following group are, in particular, for use in the treatment of cancer, more in particular for use in the treatment of cancers of hormone-dependent organs, even more in particular breast cancer, including TNBC, or prostate cancer:
the combination that comprises a therapeutically effective amount of EB1 and a therapeutically effective amount of one or more of enzalutamide and docetaxel;
the combination that comprises a therapeutically effective amount of EB1 and a therapeutically effective amount of one or more of doxorubicin and tamoxifen; and
the combination that comprises a therapeutically effective amount of EB1 and a therapeutically effective amount of one or more of an inhibitor of PD-1 or PD-L1.

This aspect may also be formulated as a method of treatment of cancer, which comprises administering to a subject in need thereof, including a human subject, either

(a) a combination or single pharmaceutical composition comprising a therapeutically effective amount of EB1, or a pharmaceutically or veterinary acceptable salt thereof; and one or more of the chemotherapeutic or immunotherapeutic compounds selected from the group consisting of doxorubicin, tamoxifen, enzalutamide, docetaxel, cisplatin, lapatinib, inhibitors of the programmed cell-death protein 1 and its ligand (PD-1/PD-L1), and combinations thereof, together with one or more pharmaceutically or veterinary acceptable excipients or carriers; or alternatively
b) the package or kit of parts as defined in the embodiments above.

It also forms part of the invention the use of a combination comprising a therapeutically effective amount of EB1, or a pharmaceutically or veterinary acceptable salt thereof; and one or more of the chemotherapeutic or immunotherapeutic compounds selected from the group consisting of doxorubicin, tamoxifen, enzalutamide, docetaxel, cisplatin, lapatinib, inhibitors of the programmed cell-death protein 1 and its ligand (PD-1/PD-L1), and combinations thereof, together with one or more pharmaceutically or veterinary acceptable excipients or carriers; for the preparation of a medicament for the treatment and/or prevention of cancer. In particular the medicament is for the treatment and/or prevention of a cancer of an hormone-dependent organ. More in particular, for the treatment and/or prevention of breast and prostate cancer.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention.

The following examples are provided by way of illustration, and they are not intended to be limiting of the present invention. Furthermore, the present invention covers all possible combinations of particular and preferred embodiments described herein.

### EXAMPLES

### Example 1. Synthesis of 4,6-diphenyl-1H-pyrazolo[3,4-b]pyridin-3-amine (EB1)

4,6-diphenyl-2-methoxynicotinonitrile (Journal of Heterocyclic Chemistry, 26(6), 1665-73; 1989) (0.6 mmol) is dissolved in 4 ml of 1,4-dioxane together with POBr₃ (1.34 mmol), pyridine-HBr (0.015 mmol) and H₃PO₄ (0.026 mmol). The mixture is heated under reflux for 18 hours at 120° C. The reaction is stopped by adding cold water and neutralised with NaOH (6M). The solid obtained is filtered and washed with cold water to obtain 4,6-diphenyl-2-bromonicotinonitrile in a yield of 84% and as a white solid, m. p.: 123-125° C.; ¹H NMR (400 MHz, DMSO-d₆) δ (ppm): 8.26 (s, 1H), 8.26-8.22 (m, 2H), 7.82-7.77 (m, 2H), 7.63-7.60 (m, 3H) and 7.59-7.55 (m, 3H); ¹³C NMR (100 MHz, DMSO-de) δ (ppm): 159.3, 156.2, 144.4, 135.5, 135.3, 131.3, 130.5, 129.1, 128.9, 127.7, 119.7, 116.5, 109.7; IR (KBr) vmax (cm⁻¹): 3432, 3029, 2225, 1649, 1575, 1517, 1493, 1419, 1373, 772, 747, 702, 686; MS (70 eV, El) m/z (%): 336.1 (96%), 335.1 (68%), 334.1 (100%), 333.1 (52%), 286.2 (36%), 285.2 (46%), 255.1 (69%), 254.1 (20%), 253.1 (31%), 228.1 (26%), 227.1 (55%), 100.1 (20%), 77.1 (43%), 51.0 (22%); Elemental analysis: calculated for C₁₈H₁₁BrN₂: C: 64.50%, H: 3.31%, N: 8.36%; obtained: C: 64.83%, H: 3.58%, N: 8.31%.

Next, 4,6-diphenyl-2-bromo-nicotinonitrile (0.18 mmol) and hydrazine monohydrate (0.36 mmol) are added to a 5 ml microwave vial and dissolved in 3 ml MeOH. The mixture is heated under microwave irradiation for 2 hours at 140° C. The solvent is removed under reduced pressure. The solid obtained is resuspended in MeOH, filtered and washed with cold methanol to obtain 4,6-diphenyl-1H-pyrazolo[3,4-b]pyridin-3-amine which is purified by column chromatography (Cy: AcOEt gradient 0% to 20% in 10 minutes, 20% isocratic for 5 minutes, 20% to 100% in 15 minutes). Yield 71%, yellowish solid, m. p.: 219-220° C.; ¹H NMR (400 MHz, DMSO-de) δ (ppm): 12.37 (s, 1H), 8.21-8.15 (m, 2H), 7.73-7.67 (m, 2H), 7.63-7.47 (m, 7H), 4.56 (s, 2H); ¹³C NMR (100 MHz, DMSO-de) δ (ppm): 155.5, 153.3, 147.2, 145.4, 138.9, 137.2, 129.2, 128.9, 128.8, 128.8, 128.7, 127.2, 112.4, 102.0; IR (KBr) vmax (cm⁻¹): 3423, 3297, 3193, 1737, 1623, 1591, 1567, 1525, 1401, 1354, 1292, 702; MS (70 eV, El) m/z (%): 287.1 (21%), 286.1 (100%), 285.1 (38%); Elemental analysis: calculated for C₁₈H₁₄N₄: C: 75.50%, H: 4.90%, N: 19.60%; obtained: C: 75.43%, H: 4.90%, N: 19.56%.

EB1 synthesized as illustrated in this Example 1 is the one used in the Examples 2-6 that follow.

### Example 2. EB1 Inhibitory Effect

Materials and Methods:

Kinase Activity Assay:

The kinase activity of the compound was measured by a radiometric kinase assay (³³PanQinase® Activity Assay) carried out by Proqinase (www.proqinase.com).

The assays were carried out in 96-well FlashPlates™ by PerkinElmer (Boston, Mass., USA) with a reaction volume of 50 μl. The reaction cocktail was pipetted in 4 steps: (1) 20 μl buffer, (2) 5 μl ATP solution (in H₂O), (3) 5 μl compound (in 10% DMSO) and (4) 20 μl enzyme/substrate mixture. Lastly, the cocktail contained 70 mM HEPES-NaOH pH 7.5, 3 mM MgCl₂, 3 mM MnCl₂, 3 μM sodium orthovanadate, 1.2 mM DTT, 50 μg/ml PEG₂₀₀₀₀, ATP (1 μM for MNK1 and 0.3 μM for MNK2), [γ-³³P]-ATP (approx. 1.2×1006 cpm per well), protein kinase (variable amounts depending on the batch) and substrate (2 μg/50 μl).

Reaction cocktails were incubated at 30° C. for 60 minutes. The reaction was stopped with 50 μl of 2% H₃PO₄ (v/v), the plates were sucked, they were washed twice with 200 μl of 0.9% NaCl (w/v) and the incorporation of ³³Pi (MicroBeta microplate scintillation counter, Wallac) was determined. All assays were performed with a Beckman Coulter/SAGIAN™ system.

The residual activity (in %) for each well of a particular plate was calculated using the following formula (wherein high control is the kinase activity in absence of an inhibitor and low control is the radioactivity value of the substrate in absence of a kinase).

$$Res . Activity ( \% ) = 100 \times [ ( signal of the compound - low control ) \text{/} ( high control - low control ) ]$$

To screen compounds, the residual activity of both kinases was analysed after treatment with 10 μM of the compounds.

To measure IC₅₀, residual kinase activity was analysed at 10 different concentrations in a range of 5·10⁻⁴ M to 1.5·10⁻⁹ M.

To study selectivity, residual activity was measured at 1 μM from a 320-kinase panel.

Cell Culture:

Commercially available MDA-MB-231, MDA-MB-468, MCF7 and A375M, IMR90 (IMR-90 (ATCC® CCL-186TH) cells were grown at 37° C. and 5% CO₂ in DMEM medium (Dulbecco's Modified Eagle Medium, 4.5 g/I glucose; 30 mg/I L-Glutamine (Gibco)) supplemented with 10% foetal bovine serum, 100 U/ml penicillin and 100 μg/ml streptomycin.

MCF10A cells (also commercially available) were grown in DMEM/F-12 medium (Dulbecco's Modified Eagle Medium: 3.125 g/L D-Glucosa, 365 mg/L L-Glutamine, Nutrient Mixture F-12 (Gibco)) supplemented with 10% FBS, 100 U/ml penicillin, 100 μg/ml streptomycin, 1% L-Glutamine (Cambrex), 10 ng/ml choleric toxin (Sigma-Aldrich), 0.005 mg/ml insulin (Sigma), 100 ng/ml hydrocortisone (Sigma) and 20 ng/ml EGF (Epidermal growth factor, Sigma).

MV4-11 cells were grown in IMDM medium (Iscove's Modified Dulbecco's Medium, Gibco) supplemented with 10% FBS, 1% L-glutamine, 100 U/ml penicillin and 100 μg/ml streptomycin.

In Vitro Proliferation Assays:

In 96-well plates, 5000 cells were seeded for each condition. After 24 h, the cells were treated with the compound at the corresponding concentration. The stocks of compounds were prepared in 100% DMSO at a concentration 200 times more concentrated than the treatment concentration. Thus, in all cases, the final concentration of DMSO in the medium is 0.5%. After incubation, the medium was removed and the cells were fixed with 4% PFA (100 μl per well) for 30 minutes. Two washes with PBS were performed.

Cells were stained with crystal violet (0.5% in EtOH): 100 μl were added to each well, stirred for 15 minutes, washed with water and left to dry. The content of each well was dissolved in 200 μl of 15% AcOH and the absorbance thereof was measured at 595 nm.

In Vitro Assays for Inhibition of eIF4E Phosphorylation:

Inhibition of eIF4E phosphorylation was quantified by Western Blot. Cells were seeded in 60 mm plates (700,000 cells for 24 h, 500,000 cells for 48 h and 300,000 cells for 72 h).

After 24 h, the treatments were added. The stocks of compounds were prepared in 100 DMSO at a concentration 200 times more concentrated than the treatment concentration. Thus, in all cases, the final concentration of DMSO in the medium was 0.5%.

After incubation, proteins were removed and Western Blot was performed. On ice, the plates were washed with PBS and the cells were lysed with 60 μl lysis buffer (50 mM Tris, 200 mM NaCl, 5 mM EDTA, 0.1% Triton 100×, pH 7.5 supplemented with protease inhibitors (EDTA-free Protease Inhibitor Cocktail Set III, Calbiochem) and phosphatase inhibitors (Phosphatase Inhibitor Cocktail Set II, Calbiochem)). The samples were centrifuged at 15,000 g and the supernatant was quantified by the Bradford method. The samples were all diluted to the same concentration with a lysis buffer and loading buffer (Laemmli buffer) and denatured for 5 minutes at 95° C. The proteins were separated on a 12% agarose gel (20 μg were loaded per well) and transferred to a PDVF (Polyvinylidene fluoride) membrane. β-Actin antibodies, abbreviated as “actin” in the figures (Calbiochem), eIF4E (anti-rabbit, Cell signalling), and phosphorylated-eIF4E (S209) (anti-rabbit, Cell signalling) in 5% BSA in TBST were used to study the activity of the compounds. Selectivity against MNK was analysed with ERK, p-ERK, MNK1, p-MNK1, 4EBP1 and p-4EBP1 antibodies.

Results:

The present invention relates to the use of 4,6-diphenyl-1H-pyrazolo[3,4-b]pyridin-3-amine EB1, or of a pharmaceutically acceptable salt thereof, as a selective MNK inhibitor with potential use in the treatment of triple-negative breast cancer and other referable cancers with p-eIF4E overexpression due to increased MNK activity.

As previously announced, and surprisingly, EB1 has an enzymatic IC₅₀ of 0.7 μM and an in vitro EC₅₀ of approximately 1.5 μM in triple-negative breast cancer cells (MDA-MB-231). Complete inhibition of eIF4E phosphorylation is achieved without cytotoxic effects and with high selectivity. Additionally, the results are independent of the cell line. Likewise, EB1 increases the sensitivity of MDA-MB-231 tumour cells to chemotherapy when combined with doxorubicin (or abbreviated as DOXO in this description).

These results are especially surprising and relevant when other results from the derivatives studied with various substituents included in the phenyl rings at positions C4 and C6 of the 1H-pyrazolo[3,4-b]pyridin-3-amine system have presented activities lower than those of EB1.

The results of the radiometric enzyme assay performed by Proqinase® (https://www.proqinase.com/) indicate that the residual activity of MNK1 and MNK2 kinases after treatment with 10 μM EB1 are 14% and 52%, respectively. These results indicate a certain selectivity for MNK1. In the same assay, the results obtained by the reference compounds cercosporamide and CGP57380 are, on one hand, −1% and 0 and, on the other, 21% and 39%.

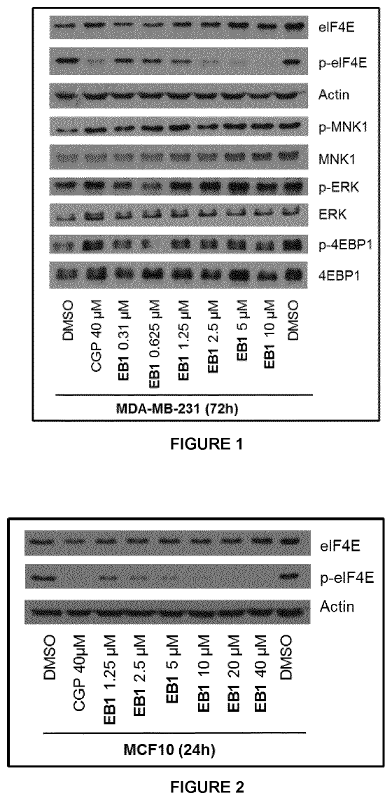

As shown in FIG. 1, EB1 inhibits eIF4E phosphorylation with in vitro EC₅₀˜1.5 μM with almost complete inhibition as of 2.5 μM. The effect is fast and lasts for at least 72 h. Moreover, in the tested times it makes no difference whether the compound is renewed every 24 h or 48 h.

EB1 significantly reduces eIF4E phosphorylation at concentrations greater than 2.5 μM. This reduction is not caused by a decrease in the total amount of eIF4E according to Western Blot analysis. This effect seems to be due to the direct inhibition of MNKs, since the treatment does not affect any of the components of the signalling pathway (FIG. 1). EB1 does not alter the phosphorylation state of MNKs. Additionally, ERK, the kinase downstream of the MAP kinase signalling pathway, is not affected in control cells and cells treated with EB1.

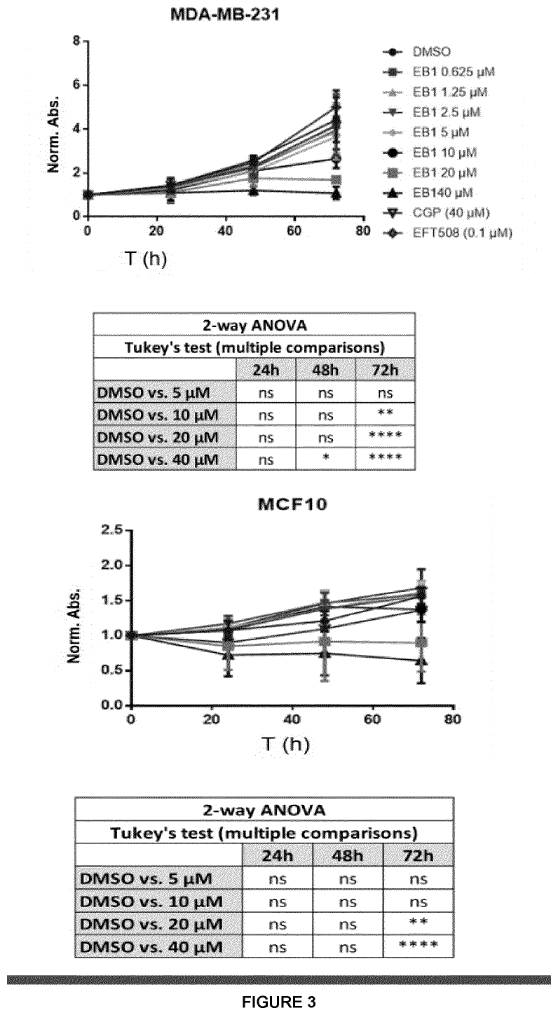

Lastly, phosphorylation of the eIF4E-binding protein (4E-BP1) is not altered by the compound either (FIG. 1). In summary, the data indicates that decreased eIF4E phosphorylation in cells treated with EB1 is due to the direct inhibition of MNK kinases. The effect of EB1 was also tested on immortalised non-tumour breast cells (MCF10) with similar results (EC₅₀=0.7 μM) (FIG. 2).

Based on the fact that knockout mice of MNK1/2 are viable, and without being bound to any theory, it is proposed that both proteins are not essential for basic cellular functions. This is also supported by the fact that shRNA-mediated deletion of both proteins does not alter cell viability and growth. Based on this data, we predict that MNK inhibitors are not expected to reduce cell growth. Therefore, it is tested whether EB1 affects cell growth in concentrations, which are sufficient to inhibit eIF4E phosphorylation.

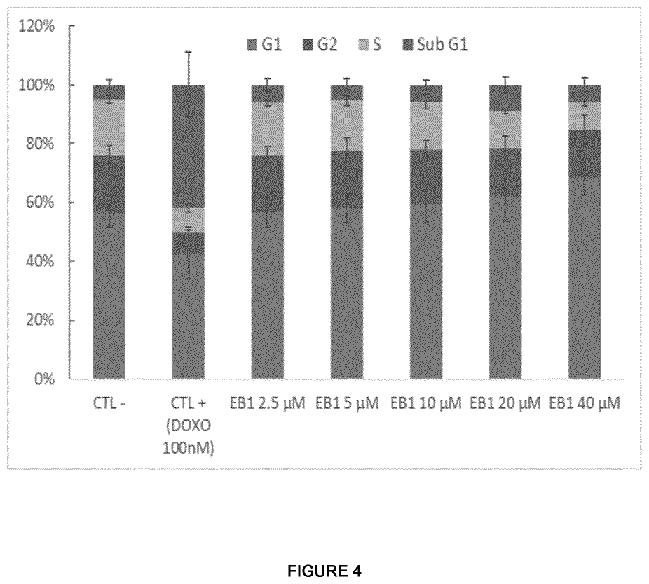

As shown in FIG. 3, in TNBC cells (MDA-MB-231) and non-tumour breast cells (MCF10), it is possible to inhibit eIF4E phosphorylation without altering cell growth and only observing a significant reduction in the number of cells at the highest concentrations.

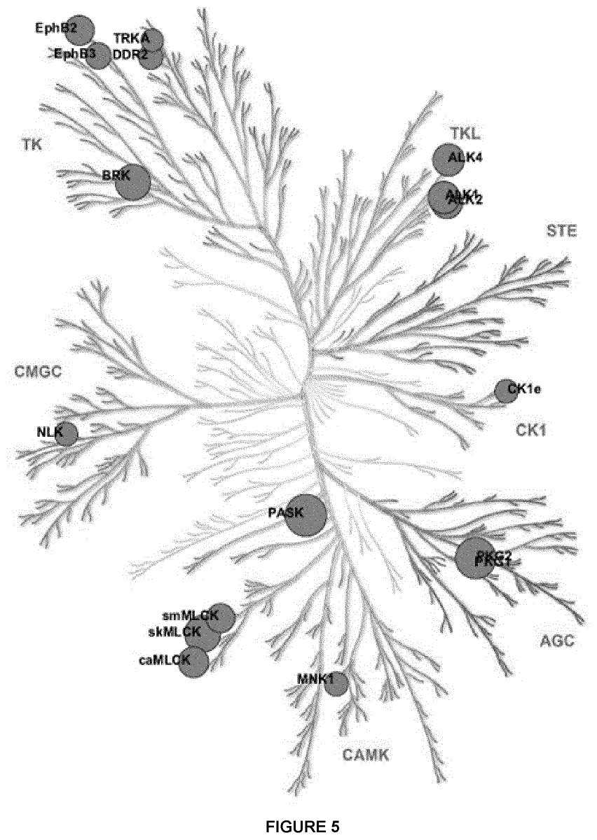

To determine how cell growth is affected at higher concentrations of EB1, a FACS analysis was performed to monitor the state of the cell cycle and the percentage of cells in subG1 as an indicator of cell death. As a positive control of cell death, cells were treated with 100 nM doxorubicin, which resulted in a sharp increase in cells in the subG1 phase. Unlike doxorubicin, concentrations up to 40 μM EB1 did not result in increased cell death. In contrast, an increase of cells could be observed in the G1 phase of the cell cycle, in a dose-dependent manner. These results, therefore, indicate that reduced cell growth at the highest concentrations of EB1 is caused by a delay in the cell cycle progression rather than by induction of cell death. Equivalent results have been described for the MNK1/2 inhibitor BAY 11433296 (FIG. 4).

In summary, EB1 does not affect the cell growth of tumour and non-tumour cells at the concentrations necessary to inhibit MNK activity, which supports the selective mode of action towards MNK and provides a first indication of a safe mode of action in future clinical studies.

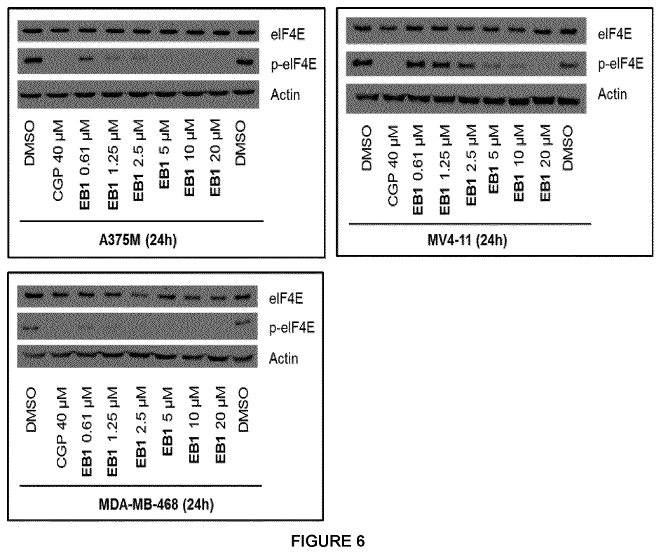

To check the selective mode of action, the activity of EB1 is assessed against a panel of different kinases (Proginase®) (FIG. 5). EB1 shows excellent selectivity at 1 μM on a 320-kinase panel as only 16 other kinases are affected in an equivalent manner. A study of the literature reveals that none of the other affected kinases is involved in pathways that affect eIF4E phosphorylation.

Reviews of previously described MNK inhibitors often describe specific effects for a given cell line. To test whether this is also the case for EB1, titration curves are performed on cell lines of different types of tumours, such as leukaemia cells (MV4-11), melanoma cells (A375M) and breast cancer cells (MDA-MB-468).

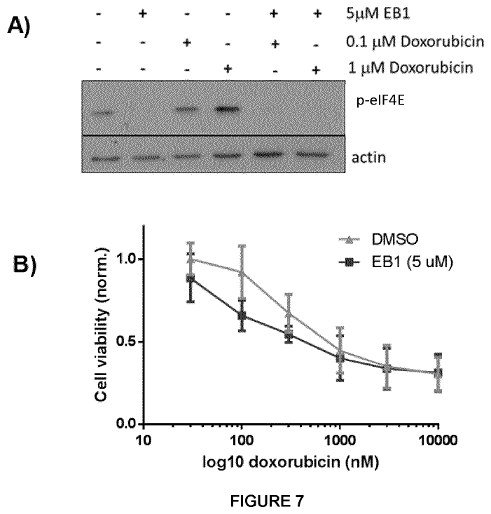

EB1 is active on all tested cell lines, which shows that the activity of said compound as an MNK inhibitor is not dependent on the cell line (FIG. 6).

Inhibition of eIF4E phosphorylation through MNK has emerged as a new strategy against cancer, especially in combination with other approved therapies. Treatment with MNK inhibitors makes it possible to sensitise tumour cells to common therapies. These new treatment schemes are also important for types of cancer in which targeted therapies are not available, such as triple-negative breast cancer. However, until now, no examples of the use of MNK inhibitors to sensitise triple-negative breast cancer to approved chemotherapies have been described.

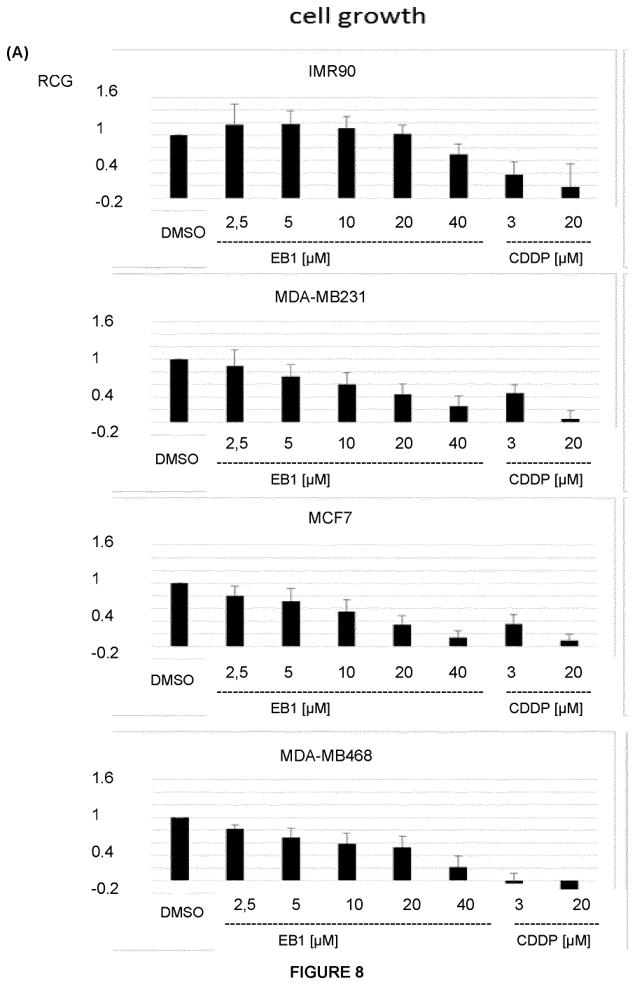

Combining EB1 with doxorubicin in MDA-MB-231 cells shows promising results. The increased eIF4E phosphorylation caused by cellular stress due to treatment with doxorubicin has been confirmed and can be reversed with 5 μM EB1 (FIG. 7A).

Additionally, the treatment together with EB1 sensitises MDA-MB-231 cells to doxorubicin, increasing the effect thereof on cell growth. The IC₅₀ of doxorubicin could be reduced in combination with EB1 from 350 nM to 225 nM (FIG. 7B).

All these results confirm the use of 4,6-diphenyl-1H-pyrazolo[3,4-b]pyridin-3-amine (abbreviated as EB1), or of a pharmaceutically acceptable salt thereof, as a selective, non-cytotoxic inhibitor of MNK1 and MNK2.

### Example 3. EB1 is not Causing Adverse Effects on Cell Growth and Cell Mortality in the IMR90 Cell Line as Model for Non-Tumor Cells

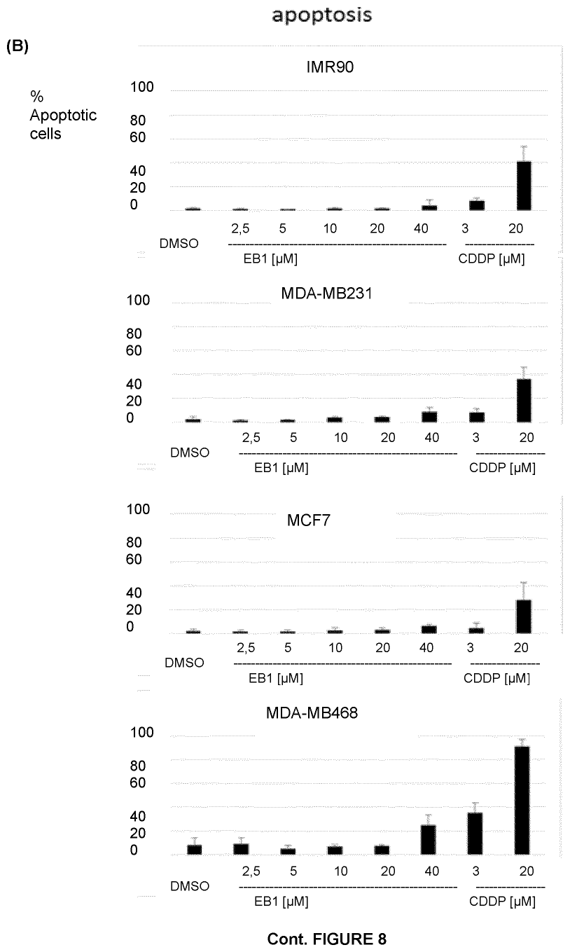

Knock-out mice of the EB1 target kinases MNK1 and MNK2 are viable (Ueda et al 2004; PMID: 15254222). Treatment of IMR90 cells, non-transformed fibroblasts, does not cause significant cell growth arrest (FIG. 8, A) and does not induce cell death (FIG. 8, B). Cell growth was measured by crystal violet assay. Cell death was determined by Propidium Iodid (PI)/Hoechst staining and the ratio of dead cell (PI positive) among all analyzed cells (Hoechst) was determined. Cis-platin treatment was included as positive control. Results indicate that EB1 is not expected to cause adverse effects on healthy cells. Minimal side effects for patients are therefore predictable.

### Example 4. EB1 Treatment Results in Increased Sensitivity to Tamoxifen

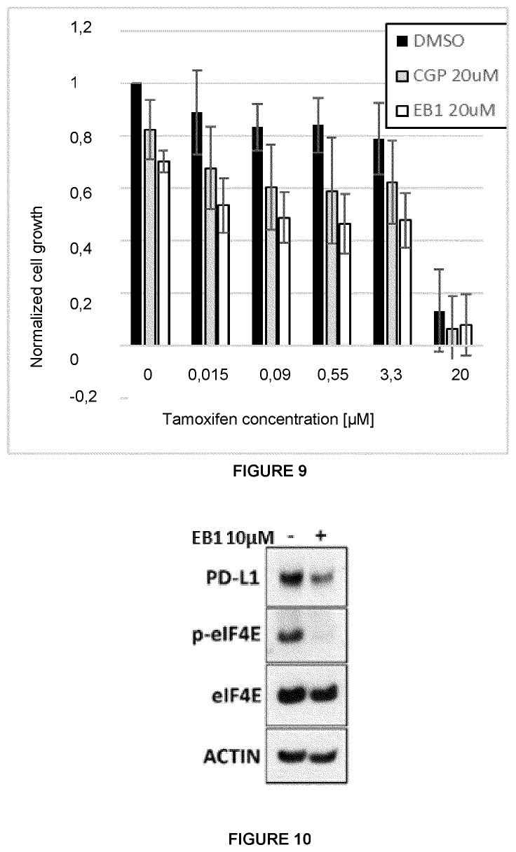

Previous studies suggest that the inhibition of MNK kinases by CGP57380 (CGP), and inhibitor that has a lot of targets, results in increased sensitivity to tamoxifen (Geter et al, 2017; PMID: 29269484) in MCF7 cells. EB1 treatment of MCF7 cells also results in increased sensitivity to tamoxifen (FIG. 9). Cell growth in the presence of EB1, tamoxifen or the combination of both inhibitors was measured by crystal violet. Data were normalized to the vehicle control.

### Example 5. EB1 Treatment Results in Reduced Levels of PD-L1 in MDA-MB231 Cells

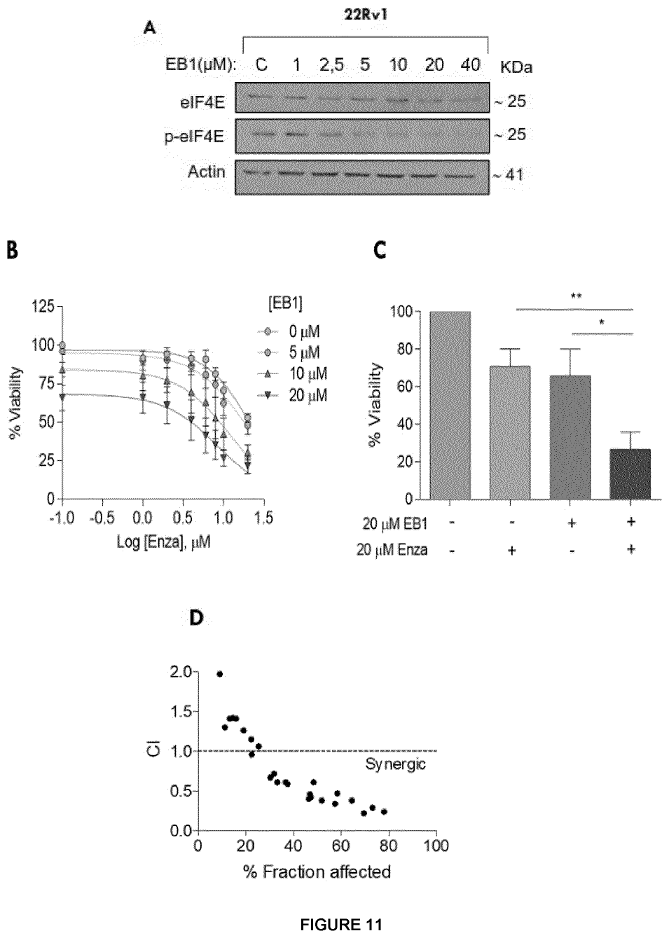

Previous studies with eFT508 in syngenic mice models suggest that inhibition of MNKs results in reduced levels of PD-L1 expression. EB1 treatment reduces levels of PD-L1 in the human breast cancer cell line MDA-MB-231. At comparable inhibition rates of eIF4E phosphorylation, EB1 is equally potent or superior to the MNK inhibitor eFT508 and CGP57380. MDA-MB-231 cells were treated for 48 h with the indicated concentrations of the inhibitors. DMSO was used as vehicle control. Protein extracts were prepared and western blot analysis were carried out. Data are depicted in FIG. 10.

### Example 6. EB1 Treatment Increases Sensitivity to Enzalutamide in Prostate Cancer Cells

Co-treatment of EB1 and Enzalutamide in the castration resistant prostate cancer cells line 22Rv1 (commercially at ATCC® CRL-2505™) resulted in a synergistic effect of the cell viability.

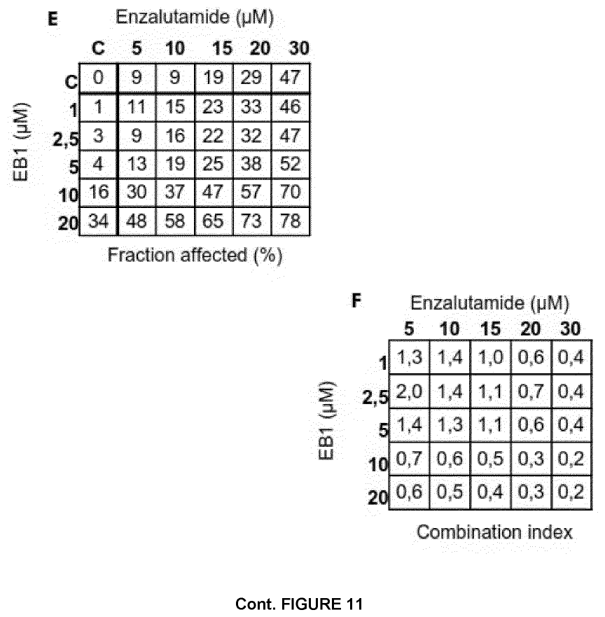

Cell viability under combined inhibition of AR and eIF4E phosphorylation was assayed. Results are shown in FIG. 11. In (A) Dose dependent inhibition of eIF4E phosphorylation was achieved after 24 h EB1 treatment in 22Rv1, determined by western blot analysis. Synergistic effect of the EB1 and Enzalutamide was investigated with 22Rv1 (B-F) cell viability assay. (B) Number of viable cells after 72 h treatment with pairwise combinations reported as percentage relative to control. (E) Cell viability data presented as a grid displaying the percentage of affected cells by each pairwise combination of drug doses. (C) Drug combinations with the strongest inhibition in cell viability (bars; mean±SEM; n=3. *p<0.05, **p<0.01 two-tailed Student's test). (F) Combination index (CI) presented as a grid for each pairwise combination of drug doses calculated by the Chou-Talalay method at non-constant ratio. (D and I) Fraction affected vs. combination index (CI) plots.

All these data allow concluding that the combination comprising EB1 or a pharmaceutically or veterinary acceptable salt thereof and enzalutamide implies a synergistic effect in terms of reduction of cell viability of prostate cancer cells.

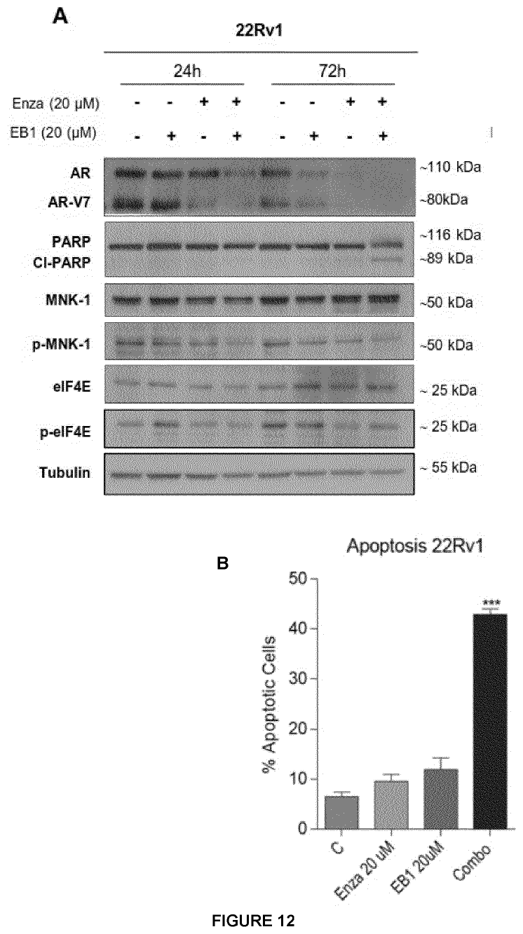

Dual inhibition of AR and eIF4E phosphorylation induces cell death via apoptosis. These results are illustrated in FIG. 12. With this aim, 22Rv1 (A) cells were treated either with vehicle (DMSO), enzalutamide, EB1 or a combination of both with the indicated concentrations for 72 h. Whole cell lysates were then prepared and subjected to western blot analysis with the indicated antibodies. A reduction of AR, its splice variant AR-V7 and eIF4E phosphorylation was observed upon treatment with EB1 alone and more pronounced with the combination. Increase in CI-PARP levels indicated apoptotic induction under the combinatory treatment. (B) Apoptosis assays of 22Rv1 cell line after treatment with the indicated concentrations of Enzalutamide, EB1 and the combination of both for 72 h, quantified by analysis of fragmented/condensed chromatin after Propidium Iodide staining. Remarcable increase in apoptotic cell population was observed upon the dual treatment. Representative Propidium Iodide and Hoechst staining images of 22Rv1 cell line were obtained, although data are not shown. Data are mean of two technical replicates from one experiment ±SEM ***p<0.001 two-tailed Student's test

Further aspects/embodiments of the present invention can be found in the following clauses:

Clause 1. The use of 4,6-diphenyl-1H-pyrazolo[3,4-b]pyridin-3-amine (EB1), or of a pharmaceutically acceptable salt thereof, as a selective, non-cytotoxic inhibitor of MAP kinase-interacting serine/threonine-protein kinase 1 (MNK1) and MAP kinase-interacting serine/threonine-protein kinase 2 (MNK2).

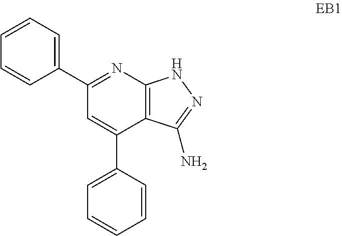 [SMILES: Nc1n[nH]c2nc(-c3ccccc3)cc(-c3ccccc3)c12]

Clause 2. The use according to clause 1, wherein the inhibition of MNK1 and MNK2 causes the inhibition of eIF4E phosphorylation.

Clause 3. 4,6-diphenyl-1H-pyrazolo[3,4-b]pyridin-3-amine (EB1), or a pharmaceutically acceptable salt thereof, for use in the treatment of triple-negative breast cancer and other referable cancers by inhibiting MNK1 and MNK2.

Clause 4. The 4,6-diphenyl-1H-pyrazolo[3,4-b]pyridin-3-amine or a pharmaceutically acceptable salt thereof for use according to clause 3, wherein other cancers refers to cancers with p-eIF4E overexpression selected from the group consisting of lung cancer, ovarian cancer, endometrial cancer, colon cancer, prostate cancer, melanoma, glioma, sarcoma, leukaemia, lymphomas and brain tumours.

Clause 5. The 4,6-diphenyl-1H-pyrazolo[3,4-b]pyridin-3-amine or a pharmaceutically acceptable salt thereof for use according to clause 3, wherein p-eIF4E overexpression is induced by treatments selected from the group consisting of chemotherapy, radiotherapy and immunotherapy.

Clause 6. The 4,6-diphenyl-1H-pyrazolo[3,4-b]pyridin-3-amine or a pharmaceutically acceptable salt thereof for use according to clause 3, wherein the use for the treatment of triple-negative breast cancer and other cancers with p-eIF4E overexpression is achieved by the sensitisation that it confers with regards to chemotherapy treatments, immunotherapy treatments, radiotherapy treatments and other drugs for the treatment of cancer.

Clause 7. The 4,6-diphenyl-1H-pyrazolo[3,4-b]pyridin-3-amine or a pharmaceutically acceptable salt thereof for use according to clause 6, wherein chemotherapy treatments include doxorubicin, and immunotherapy treatments include programmed cell death protein 1 and the PD-1/PD-L1 ligand thereof.

### CITATION LIST

### Patent Literature

US 2010/0113415
WO 2011/019780
WO 2015/004024
DE 2160780

### Non Patent Literature

Clin Cancer Res. 2007; 13:4429-4434
Nat Rev Clin Oncol. 2016; 13:674-690
Oncologist. 2011; 16 Suppl 1:1-11
Breast Cancer Res. 2010; 12:212
J. Am. Soc. Clin. Oncol. 2008; 26:1275-1281
Lancet Lond. Engl. 2014; 384:164-172
J. Am. Soc. Clin. Oncol. 2012; 30:1796-1804
Med. Oncol. Northwood Lond. Engl. 2017; 35:13
Clin Transl Oncol. 2014; 16:937-1113.
Oncotarget. 2015; 6:24092-24104
PLoS ONE. 2014; 9:e89220
J Cancer Res Clin Oncol. 2016; 142:2309-2317
J Neurooncol. 2016; 131:485-493
Virchows Arch. 2015; 467:667-673
Int J Clin Exp Pathol. 2015; 8:3955-3962
J Invest Dermatol. 2015; 135:1358-1367
Oncogene. 2015; 34(16):2032-2042
PLoS ONE. 2015; 10(4):e0123352
Mol Cell Biol. 2004; 24(15):6539-6549
Cancer Biol Ther. 2015; 16(5):648-656
Cancer Res. 2011; 71:1849-1857
Biochem. J. 2007; 408(3):297-315
Curr Med Chem. 2017; 24(28):3025-3053.
Oncotarget. 2012; 3:118-131; Chem Biol. 2014; 4:441-452
Curr Med Chem. 2017; 24(28):3025-3053
J Med Chem. 2018; 61(8):3516-3540
Cancer Lett. 2017; 390:21-29
Geter et al, 2017; PMID: 29269484

1. A method of treatment and/or prevention of a cancer of a hormone-dependent organ selected from breast cancer, prostate cancer, ovarian cancer and endometrial cancer, which comprises administering to a mammal subject in need thereof, a therapeutically effective amount of 4,6-diphenyl-1H-pyrazolo[3,4-b]pyridin-3-amine or a pharmaceutically or veterinary acceptable salt thereof, wherein the subject in need thereof includes a human subject.

2. The method according to claim 1, wherein the cancer of the hormone-dependent organ is selected from breast cancer and prostate cancer.

3. The method according to claim 2, wherein the breast cancer is triple negative breast cancer.

4. The method according to claim 2, wherein the cancer of the hormone-dependent organ is prostate cancer.

5. The method according to claim 1, wherein the treatment comprises administering the therapeutically effective amount of 4,6-diphenyl-1H-pyrazolo[3,4-b]pyridin-3-amine, or the pharmaceutically or veterinary acceptable salt thereof, in combination with one or more compounds selected from compounds for chemotherapy and compounds for immunotherapy.

6. The method according to claim 5, wherein the chemotherapeutic compounds are selected from the group consisting of doxorrubicin, tamoxifen, enzalutamide, docetaxel, cisplatin, lapatinib, and combinations thereof.

7. The method according to claim 5, wherein the compounds for immunotherapy are selected from the group consisting of inhibitors of the programmed cell-death protein 1 and its ligand (PD-1/PD-L1).

8. A combination comprising:
a) a therapeutically effective amount of 4,6-diphenyl-1H-pyrazolo[3,4-b]pyridin-3-amine, or a pharmaceutically or veterinary acceptable salt thereof; and
b) a therapeutically effective amount of one or more chemotherapeutic or immunotherapeutic compounds selected from the group consisting of doxorubicin, tamoxifen, enzalutamide, docetaxel, cisplatin, lapatinib, inhibitors of the programmed cell-death protein 1, a ligand of inhibitors of the programmed cell-death protein 1 (PD-1/PD-L1), and combinations thereof.

9. The combination according to claim 8, comprising 4,6-diphenyl-1H-pyrazolo[3,4-b]pyridin-3-amine, or the pharmaceutically or veterinary acceptable salt thereof, and one of or a combination of enzalutamide and docetaxel.

10. The combination according to claim 8, comprising 4,6-diphenyl-1H-pyrazolo[3,4-b]pyridin-3-amine, or the pharmaceutically or veterinary acceptable salt thereof, and one of or a combination of doxorrubicin and tamoxifen.

11. The combination according to claim 8, comprising 4,6-diphenyl-1H-pyrazolo[3,4-b]pyridin-3-amine, or the pharmaceutically or veterinary acceptable salt thereof, and one of or a combination of an inhibitor of the programmed cell-death protein 1 and its ligand (PD-1/PD-L1).

12. A single pharmaceutical or veterinary composition which comprises, together with one or more pharmaceutically or veterinary acceptable excipients or carriers, a therapeutically effective amount of:
a) 4,6-diphenyl-1H-pyrazolo[3,4-b]pyridin-3-amine, or a pharmaceutically or veterinary acceptable salt thereof; and
b) one or more chemotherapeutic or immunotherapeutic compounds selected from the group consisting of doxorubicin, tamoxifen, enzalutamide, docetaxel, cisplatin, lapatinib, inhibitors of the programmed cell-death protein 1, a ligand of inhibitors of the programmed cell-death protein (PD-1/PD-L1), and combinations thereof.

13. (canceled)

14. The method according to claim 2, wherein the treatment comprises administering the therapeutically effective amount of 4,6-diphenyl-1H-pyrazolo[3,4-b]pyridin-3-amine, or the pharmaceutically or veterinary acceptable salt thereof, in combination with one or more compounds selected from compounds for chemotherapy and compounds for immunotherapy.

15. The method according to claim 3, wherein the treatment comprises administering the therapeutically effective amount of 4,6-diphenyl-1H-pyrazolo[3,4-b]pyridin-3-amine, or the pharmaceutically or veterinary acceptable salt thereof, in combination with one or more compounds selected from compounds for chemotherapy and compounds for immunotherapy.

16. The method according to claim 4, wherein the treatment comprises administering the therapeutically effective amount of 4,6-diphenyl-1H-pyrazolo[3,4-b]pyridin-3-amine, or the pharmaceutically or veterinary acceptable salt thereof, in combination with one or more compounds selected from compounds for chemotherapy and compounds for immunotherapy.
